SEQUENCE LISTING (5 sequences)

SEQ ID NO: 1 - protein, 191 aa, Artificial Sequence
  Synthetic peptide; N terminal amino acid is Met; Native pituitary bSt contains an N terminal Ala
MFPAMSLSGLFANAVLRAQHLHQLAADTFKEFERTYIPEGQRYSIQNTQVAFGFSETIPAPTCKNEAQQKSDLELLRISLLLIQSWLGPLQFLSRVFTNSLVFGTSDRVYEKLKDLEEGILALMRELEDGTPRAGQILKQTYDKFDTNMRSDDALLKNYGLLSCFAKDLHKTETYLRVMKCRRFGEASCAF

SEQ ID NO: 2 - protein, 191 aa, Bos Taurus
  natural bovine pituitary somatotropin
AFPAMSLSGLFANAVLRAQHLHQLAADTFKEFERTYIPEGQRYSIQNTQVAFGFSETIPAPTCKNEAQQKSDLELLRISLLLIQSWLGPLQFLSRVFTNSLVFGTSDRVYEKLKDLEEGILALMRELEDGTPRAGQILKQTYDKFDTNMRSDDALLKNYGLLSCFAKDLHKTETYLRVMKCRRFGEASCAF

SEQ ID NO: 3 - protein, 20 aa, Artificial Sequence
  Synthetic peptide; N terminal amino acid is Met; Native pituitary bSt contains an N terminal Ala; this peptide was used as an antigen in the production of monoclonal antibodies against rbSt
MFPAMSLSGLFANAVLRAQH

SEQ ID NO: 4 - protein, 9 aa, Artificial Sequence
  Synthetic peptide; N terminal amino acid is Met; this peptide was synthetically conjugated to bovine serum albumin (BSA) using malemide activated carrier protein; in order to conjugate this peptide to BSA, the Cys terminal end includes a -NH2
MFPAMSLSC

SEQ ID NO: 5 - protein, 583 aa, Bos Taurus
  natural bovine serum albumin
DTHKSEIAHRFKDLGEEHFKGLVLIAFSQYLQQCPFDEHVKLVNELTEFAKTCVADESHAGCEKSLHTLFGDELCKVASLRETYGDMADCCEKQEFERNECFLSHKDDSFDLFKLKFDFNTLCDEFKADEKKFWGKYLYEIARRHFYFYAFELLYYANKYNGVFQECCQAEDKGACLLFKIETMREKVLASSARQRLRCASIQKFGERALKAWSVARKSQKFPKREFVEVTKLVTDLTKVHKECCHGDLLECADDRADLAKYICDNQDTISSKLKECCDKFLLEKSHCIAEVEKDAIPENLPPLTADFAEDKDVCKNYQEAKDAFLGSFLYEYSRRHPEYAVSVLLRLRKEYEATLEECCAKDDPHACYSTVFDKLKHLVDEFQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

(19) United States
(12) Patent Application Publication
(10) Pub. No.: US 2020/0319158 A1
(43) Pub. Date: Oct. 8, 2020

(54) Lateral flow immunoassay (LFIA) system for measuring reombinant bovine somatotrophin (rBST) in whole milk and other dairy products

(71) Applicant: Nelson-Jameson, Inc., Marshfield, WI (US)

(72) Inventor: Ronald Gorewit, Slaterville Springs, NY (US)

(73) Assignee: Nelson-Jameson, Inc., Marshfield, WI (US)

(21) Appl. No.: 16/501,750

(22) Filed: Jun. 3, 2019

Related U.S. Application Data
(60) Provisional application No. 62/763,238, filed on Jun. 6, 2018.

(51) Int. Cl. G01N 33/04

(52) U.S. Cl. CPC G01N 33/04

(57) ABSTRACT

The present invention discloses a competitive lateral flow immunoassay for detection of recombinant bovine somatotropin (rbSt) in bovine milk or a milk product or extract thereof from lactating dairy cows treated/injected with rbSt. The rbSt (analyte) in milk or its derivatives, is added to a sample pad. The rbSt mouse or rat monoclonal antibody (primary antibody) is complexed to gold nano-particles and dried down on the conjugate pad. This complex (gold conjugated to monoclonal anti-rbSt) then flows to the test and control lines by capillary action through their respective channels, as a result of the osmolarity of the wicking pad. A solution of rbSt-BSA (Bovine Serum Albumin) is striped/bound to the test line. As the conjugate is rehydrated by the sample and flows down the strip, rbSt, if present in the test sample (milk), competes with rbS-BSA at the test line for binding to the gold labeled rbSt monoclonal primary antibody. If rbSt is present in the sample, the test line signal will be lighter than if there was no rbSt. The test line signal is inversely proportional to the rbSt concentration in the milk sample. The control line contains a second antibody (IgG) directed against the primary monoclonal antibody bound to the gold particle. The test line and control lines are analyzed by a reader containing a photodiode (leelu reader.)

1. Recombinant bovine growth hormone (rbSt) is isolated from Posilac (Monsanto/Bayer/Elanco/Union Agener). Posilac is a viscous material. Therefore, a solvent extraction is needed to isolate rbSt. The solvent extraction is performed using water.

2. When using water to extract rbSt from Posilac, the supernatant is run through a gel exclusion spin column to remove unwanted protein bands. Thus, purifying the rbSt from Posilac.

3. SDS-PAGE is used to isolate rbSt from the extraction. Based on UV280 analysis, water extraction and using a size exclusion spin column results in approximately 36 mg of purified rbSt (see lane B, FIG. 1).

The extracted purified rbSt from Posilac contains the 20 amino acid sequence as set forth in SEQ ID NO 3: MFPAMSLSGLFANAVLRAQH, which confirms the presence of rbSt. The rbSt sequence begins with a methionine (“M”). The amino acid sequence for native pituitary (pbSt) begins with an “A”. Purified rbSt is therefore used for monoclonal antibody production.

1) Posilac is a viscous material. Therefore, it is necessary to perform a solvent extraction screen using water, dimethyl sulfoxide (DMSO), dithiothreitol (DTT), methanol and ethanol. Water performed best for extracting rbSt from Posilac.

2) Using water to extract rbSt from Posilac, the supernatant was run through a gel exclusion spin column to remove unwanted protein bands. Thus, purifying the rbSt from Posilac.

3) SDS-PAGE was performed on the Posilac extract. Purified rbSt was then obtained (FIG. 1). Based on UV280 analysis, water extraction and using a size exclusion spin column resulted in approximately 36 mg of purified rbSt (see lane B).

4) The purified protein contained the 20 amino acid sequence as set forth in SEQ ID NO 3: MFPAMSLSGLFANAVLRAQH, which confirmed rbSt. The rbSt sequence begins with a methionine (“M”). The amino acid sequence for native pituitary bSt begins with an “A” (AFPAMSLSGLFANAVLR). The purified rbSt was used for monoclonal antibody production.

Standards for LFIA Development

Highly purified bovine pituitary somatotropic hormone (pbSt) was obtained from Harbor-UCLA Research and Education Institute (a.k.a. Los Angeles Biomedical Research Institute. The sequence of pbSt is set forth in SEQ ID NO 2. rbSt conjugate was obtained from Abgent. (Synthesis ID: G050732, Sequence H-MFPAMSLSC-NH2 as set forth in SEQ ID NO 4, Final Purity>96%).

Monoclonal Antibody Production Against rbSt

The sensitivity of the Lateral Flow Immunoassay (LFIA) is to a large extent dependent on the affinity of a specific antibody or antibody pair. Specific antibodies (primary antibodies) can be labelled to provide a sensitive response; however, a high concentration of antibody is necessary. When secondary anti-species antibodies are labelled, the primary antibodies can be titrated for optimal response. Moreover, the affinity of the primary antibodies may decrease upon conjugation to the label (gold nanoparticles, etc.), diminishing the sensitivity. This phenomenon is comparable to immobilization of an enzyme in a reactor. Steric hindrance may hamper the antigen-antibody interaction as well. The dependency of the affinity on labelling primary antibody cannot be predicted.

In general terms, monoclonal antibodies produced in mice, are often preferred for most lateral-flow immunoassay systems. However, polyclonal antibodies, such as those produced in sheep, rabbits or goats, can give excellent results in many applications. Commercial issues, as well as antigen-dependent issues also come into play. Supplies of polyclonals are not necessarily as stable, as animals may die, and the antibodies are of course subject to natural variability. Unless the under development is for something present at concentrations higher than nanomolar, an antibody with high affinity is required.

The patent application herein, describes production of a monoclonal antibody (mab) capable of detecting recombinant bovine somatotropin (rbSt), a synthetic analogue of pituitary bovine somatotropin (pbSt) with a singular difference of 1 amino acid. The sequence of rbSt and pbSt according to the present application are set forth as SEQ ID NO 1 and SEQ ID NO 2, respectively. This level of differentiation requires highly specific antibodies. As such, a series of mab antibody clones were developed in mice and rats.

Immunogen for production of mouse (M) and rat (R) monoclonal antibodies was purified rbSt from Posilac, containing a water-soluble adjuvant. Animals were injected with antigen-adjuvant initially, followed by four “boost” injections on a biweekly basis.

After the last biweekly injection of rbSt, spleen cells (B cells) were isolated from injected animals. Antibody producing spleen cells were fused with myeloma cells using polyethylene glycol (PEG). The fusion partner used for mice was the SP2/0 mouse myeloma cell line. The fusion partner for rats was the YB 2/0 rat myeloma cell line. HAT (hypoxanthine-aminopterin-thymidine) medium was added to the cultured B cells. Time was allowed for the unfused B cells to die.

Media from fused cells was screened using an ELISA format for positive specific antibody binding, using both purified rbSt and synthetic rbSt peptide-BSA (bovine serum albumin) conjugate. The sequence of bovine serum albumin (BSA) according to the present application is set forth as SEQ ID NO 5.

As part of the antibody development, over 92 clones (rat and mice) of antibody were isolated and screened for reactivity with rbSt, lack of reactivity with pbSt, and cross-screened against Posilac all in an ELISA format. Of those clones, 3 rat and 4 mice monoclonal were identified as viable candidates to test in a lateral flow format.

Clones obtained (M60, Mouse 60) and R32, Rat 32) specifically bound to both rbSt isolated from Posilac and rbSt peptide-BSA conjugate. Neither M60 or R32 bound to bovine pituitary somatotropin (pbSt) or pbSt peptide-BSA.

The best antibody pairing requires the greatest visual difference in signal between the pbSt and rbSt binding (no binding to pbSt versus clear strong binding to rbSt) Antibody M60, showed a strong affinity toward rb St binding.

Nitrocellulose Membranes

Nitrocellulose membranes are a critical factor in LFIA development. CN95 and FF80 Plus nitrocellulose membranes were selected for the LFIA described herein.

Evaluation of Test Line Concentrations Using rbSt Peptide

Test line concentrations of rbSt free peptide were performed at 125 ng/ml, 62.5 ng/ml, 31.25 ng/ml, and 15.125 ng/ml. Dilutions were made from a stock solution of 38 mg/ml rb St in phosphate buffered saline to achieve concentrations of 125 ng/ml, 62.5 ug/ml, 31.25 ng/ml and 15.125 ng/ml.

Seventy-five microliters of rbSt peptide was added to each strip. Strips were incubated at room temperature and read using a Leelu reader. FIGS. 2 and 3 show the average test line peak intensity and average test line/control line peak intensity, respectfully. After reading the strip using a Leelu reader, the signal can be seen at 31.25 ng/ml rbSt

Lateral Flow Immunoassay Using Fresh Milk from a Lactating Cow, with the Addition of rbSt (Spiked) to Determine Level of Detection (LOD) in the Sample Matrix.

Materials:

| Material | Supplier | Description |
|---|---|---|
| Sample Matrix | Nelson Jameson | Raw Milk |
| rBST Free Peptide | Abgent | rBST Alone; ID: H011143, |
| | | purity ≥95%, |
| | | Concentration: 3.8 mg/ml, |
| | | Lot: PT0111719-3 |
| | | Sequence set forth in |
| | | SEQ ID NO 1 |
| Test line Strip | Lumos | AE100; rBST-BSA 1:4 0.0343 |
| | | mg/ml, 3 ul |
| | | M60 GNS/ |
| Sample Pad | Whatman | VF2, 16 mm |
| Absorbent Pad | Whatman/GE | Grade #470, 17 mm |
| Treated | Ahlstrom | Grade 8951; |
| Conjugate Pad | | Treated with CPB: 50 mM |
| | | Borate pH 8.6, 1% BSA, |
| | | 1% Surfactant 10 G, |
| | | 0.2% Tween 20, 10 mm |

Methods:

rBST Free Peptide Standard Preparation: Dynamic Range—125 ng/ml, 62.5 ng/ml, 31.25 ng/ml, 15.125 ng/ml
rBST concentration=3.8 mg/ml in DI H₂O
Dilute rBST in raw milk to above concentrations
(1 mg/ml) (0.05 ml)=(Xml)(3.8 mg/ml)
1 mg/ml 0.0132 ml×1000=13.2 ul rBST 3.8 mg/ml+36.8 ul of raw milk
250 ug/ml (10 ug/ml) (1 ml)=(Xml)(1 mg/ml)
10 ul rBST 1 mg/ml+990 ul raw milk
125 ng/ml/1000 ng/ml (125 ng/ml) (2 ml)=(Xml)(10 ug/ml)
250 ul rBST 110 ug/ml+raw milk 1:2 Serial Dilutions 2 ml:
62.5 ng/ml−1 ml 1000 ng/ml+1 ml raw milk
31.25 ng/ml−1 ml 500 ng/ml+1 ml raw milk
15.125 ng/ml−1 ml 500 ng/ml+1 ml raw milk
0—raw milk
Pipette 75 ul of sample onto each strip

As seen in FIGS. 4 and 5, the level of detection of rbSt in milk was as low as 15 ng/ml

Summary of Lateral Flow Immunoassay Components and Conditions for rbSt detection in milk are shown in Table 1

TABLE 1

| | | |
|---|---|---|
| Membrane | Material | Whatman/GE AE 100 |
| | Blocker Buffer | N/A |
| | Test Line | rBST-BSA 1:4 0.03 mg/ml |
| | Control Line | Goat anti-Mouse 1.0 mg/ml |
| | Dimensions | [TL] 10\mm from bottom edge of |
| | | membrane to center of line |
| | | [CL] 16 mm from bottom edge of |
| | | membrane to center of line |
| | Striping | Length: 300 mm |
| | Parameters | Speed: 50 mm/s |
| | (Frontline) | Acceleration: 1000 mm/s2 |
| | | X Start: 0 mm |
| | | Y Start 50 mm |
| | | Z up: 0 mm |
| | | Z down: 54.5 mm |
| | | Dispense Rate: 0.7 ul/cm |
| Conjugate | Antibody | M60 conjugated to 150 nm Gold |
| | Loading | Nanoshells @ 15 ug/ml OD 20 |
| | Concentration | |
| | Conjugation pH | N/A |
| | Buffers | GNS Block Buffer |
| | | GNS Resuspension Buffer |
| | | GNS Reaction Buffer |
| | Conjugate OD | OD = 40 |
| | Sugars | 10% Sucrose, 5% Trehalose |
| | Conjugate | Ahistrom 8951, 10 mm |
| | Pad | |
| | Treatment | |
| | Dispense Rate | 3 ul/strip or 7.5 ul/cm |
| Sample Pad | Material | Millipore CO48, 16 mm |
| | Treatment | N/A |
| Absorbent Pad | Material | Whatman Grade #479, 17 mm |
| Backing Card | Material | Lohmann hCG Backing Card |
| | | P/N: LC-5897 |
| | Buffer | 1X PBS pH 7.4, 0.1% Tween 20 |
| Standards | Buffer | Abgent; ID: H011143, LOD 15 ng/ml |
| | rbSt Free Peptide | |

Lateral Flow Assay Protocol for Detecting rbSt in Milk and Milk Derived Products

Materials Needed:

1. Test Strips

2. Leelu Reader

3. Calibrators: Negative, Low, Medium and High spiked buffers corresponding to (0, 30, 60 and 120 ng/mL rBST).

4. Timer: to time assay run time of 15 min

Protocol:

1. Load Lumos Reader Program on your Laptop/Computer

2. Ensure the Program opens correctly prior to beginning the testing.
a. Make sure to have the YAML file associated with this assay loaded.

3. Open the foil pouches, remove the test strips and lay them with the flat, smooth side now.
Minimum 1 strip per assay but recommended 2-3 strips per condition.

4 To begin testing, add s 75 ul of standards and (milk) sample to be tested, on separate strips.
Add sample to the sample pad (FIG. 6)

5. Start a 15 minute timer after adding the last sample or standard.

6. At 15 minutes, read the strip with the Leelu Reader to obtain the relative Test Line and
Control Line intensities.

The “membrane” shown in FIG. 6 contains the test and control lines. The absorbent pad is otherwise known as the “wicking pad”. The test line contains rbSt-BSA conjugate that has been preabsorbed and dried to promote competition between the analyte (rbSt) in the sample and rbSt-BSA for binding the gold labeled monoclonal antibody conjugate. Thus, it is a competitive lateral flow immunoassay.

### BACKGROUND OF INVENTION

Bovine growth hormone is a protein synthesized, stored and secreted from the anterior pituitary gland. Administration of bovine growth hormone isolated from cattle at slaughter (bovine pituitary somatotropin (pbSt) can increase milk production when injected into well fed lactating cows. It is considered to be a galactopoietic hormone (enhances an established lactation). Its effects are mediated by the insulin-like growth factor (IGF-1), which is significantly elevated in response to pb St administration.

Until the 1980's, the use of pituitary derived pbSt was limited to research in animal endocrinology experimentation. During this time period, research on the structure and function of the hormone increased. Genentech, a biotech company, succeeded in cloning the gene for pbSt. Sometime after the successful gene cloning, they licensed their cloning patents to Monsanto. Shortly after this, a collaborative effort was begun to develop a recombinant version of the hormone that could be injected into lactating cows. The two companies used their genetic engineering techniques to clone the bSt gene into E. coli bacteria for mass production of the recombinant hormone. The bacteria could then be grown in bioreactors, to produce recombinant bovine somatotropin (rbSt), which was then purified from fermentation media to produce the injectable hormone. They published their first field trial results in 1981 showing that rbSt is galactopoietic when injected into lactating cows.

It should be noted that the chemical structure of rbSt is not identical to native bovine pituitary growth hormone (bSt). It differs in its amino acid structure. The amino acid methionine is added to the n-terminal end of the hormone.

Lilly, American Cyanamid, Upjohn, and Monsanto all submitted applications to market rbSt. to the U.S. Food and Drug Administration (FDA). The FDA completed its review of the human safety component of these applications in 1986. The FDA eventually decided that milk from rbSt-treated cows was safe for human consumption. These companies strongly urged the FDA to approve the use of rbSt. The FDA approved Monsanto's application in 1993. Monsanto launched rbSt, brand-named Posilac, in 1994. Lilly later purchased the rights to market Posilac to dairies in the United States and other countries allowing its use.

As yet, no one has reported the presence of rbSt in raw milk from rbSt treated cows. Various animal scientists and physicians believe that raw milk does not contain rbSt, theorizing that it is either denatured during pasteurization or destroyed after consumption in the gastrointestinal system. At present, there is scientific proof that raw and pasteurized milk contain antibodies to rbSt. It is unknown where these antibodies are produced, systemically or in the immune system of the mammary gland. One could argue that rbSt is in fact in the milk, as it may be bound to antibodies in situ within raw and pasteurized milk. Another possibility is that past researchers used obsolete or less sensitive technology to determine its presence in milk. Specific monoclonal antibodies (mabs) were not developed for rbSt when the FDA made its rulings on rb St.

There are presently a host of mabs against various antigenic sites (epitopes) that may recognize antibody bound rbSt. Yet, dairy products are currently sold with labels stating they are “rbSt free” or that they have not been produced from rbSt treated cows. The FDA has stated, to label dairy products “rbSt free” is in fact misleading, but dairy producers continue to label their products as “rbSt free,” in milk or processed dairy products. Therefore, there is a need for a simple, rapid, specific and low-cost test that will identify the presence of rbSt in milk or processed dairy products.

Posilac acts by stimulating the production of a biochemical called insulin-like growth Factor 1 (IGF-1) during its action, thus promoting milk production in the lactating cow. Blood serum concentrations of IGF-1 are significantly elevated after cows are injected with Posilac, “The evidence for potential harm to humans from rbSt milk is inconclusive. It is not clear that drinking milk produced using rbSt significantly increases IGF-1 levels in humans or adds to the risk of developing cancer. Of greater concern is the fact that milk from rbSt-treated cows has higher levels of IGF-1, a hormone that normally helps some types of cells to grow. Several studies have found that IGF-1 levels at the high end of the normal range may influence the development of certain tumors. Some early studies found a relationship between blood levels of IGF-1 and the development of prostate, breast, colorectal, and other cancers, but later studies have failed to confirm these reports or have found weaker relationships. While there may be a link between IGF-1 blood levels and cancer, the exact nature of this link remains unclear.

Some studies have shown that adults who drink milk have about 10% higher levels of IGF-1 in their blood than those who drink little or no milk. There have been no direct comparisons of IGF-1 levels in people who drink ordinary cow's milk vs. milk stimulated by rbSt. This is a great concern to consumers of milk and other dairy products derived from rbSt treated dairy cows. In fact, various dairy product companies in the Midwestern United States will soon reject bulk milk from dairy farmers using rbSt.

It is known that rbSt treated cows show changes in the bioactivity and structure of a protein called fatty acid binding protein (FABP). Changes in this protein may influence the processing of cheese and other dairy products.

At present, there is no specific/reliable commercial test for rbSt and/or IGF-1 in whole milk from dairy cows treated with (Posilac) rbSt. The test described in this application can be readily applied at cow side, dairy plant or milk product production plant.

Immunological Based Assays for Bovine Hormones in Milk

Progesterone

Early diagnosis of pregnancy in cows has been one of the most important tasks in agriculture. Dairy farmers must achieve pregnancy in their cattle because it is necessary to establish lactation and subsequent milk production. There are only a few published studies that describe the detection of hormones in milk they relate to the identification and quantification of progesterone in milk. In fact, the first LFIA method for detecting a low molecular weight compound/hormone or hapten was developed for the steroid hormone progesterone. LFIA kits for detecting progesterone in milk are commercially available. The limit of detection for the LFIA is as low as 2 ng/ml. Assay time is about 15 minutes.

At the present time, there is no rapid direct method for quantifying rbSt (free or antibody bound) concentrations nor IGF-1 in milk of cows treated with rbSt. Based on the successful application of LFIA for detecting the presence of milk progesterone, development of a LFIA for rb St and in milk will prove to be an asset rather than a liability to the dairy farmer and consumers of their products. Milk products will then be honestly labelled as “rbSt free.

### BRIEF SUMMARY OF THE INVENTION

This application focuses on a lateral flow immunoassay for detection of recombinant bovine somatotropin (rbSt) in bovine milk or a milk product or extract thereof from lactating dairy cows treated/injected with rbSt. The rbSt (analyte) in milk or its derivatives, is added to a sample pad. The rbSt mouse or rat monoclonal antibody (primary antibody) is complexed to gold nano-particles and dried down on the conjugate pad. This complex (gold conjugated to mab anti-rbSt) then flows to the test and control lines by capillary action through their respective channels, as a result of the osmolarity of the wicking pad. A solution of rbSt-BSA (Bovine Serum Albumin) is striped/bound to the test line. As the conjugate is rehydrated by the sample and flows down the strip, rbSt, if present in the test sample (milk), competes with rbS-BSA at the test line for binding to the gold labeled rbSt monoclonal primary antibody. Thus, it is a competitive lateral flow assay. If rbSt is present in the sample, the test line signal will be lighter than if there was no rbSt. The test line signal is inversely proportional to the rbSt concentration in the milk sample. The control line contains a second antibody (IgG) directed against the primary monoclonal antibody bound to the gold particle. The test line and control lines are analyzed by a reader containing a photodiode (leelu-reader.)

### DESCRIPTION OF FIGURES

FIG. 1 describes the purification procedure of rbSt from Posilac.

FIG. 2 describes the Leelu readings for average test line peak intensity of various concentrations of rbSt from Posilac.

FIG. 3 describes the Leelu readings for average test line/control like peak intensity of various concentrations of rbSt from Posilac.

FIG. 4 describes the average test line peak intensity Leelu readings for rbSt spiked milk.

FIG. 5 describes t describes the Leelu readings for test line/control line peak intensity Leelu spiked milk.

FIG. 6 describes the rbSt lateral flow immunoassay test strip “for detecting rbSt in milk.

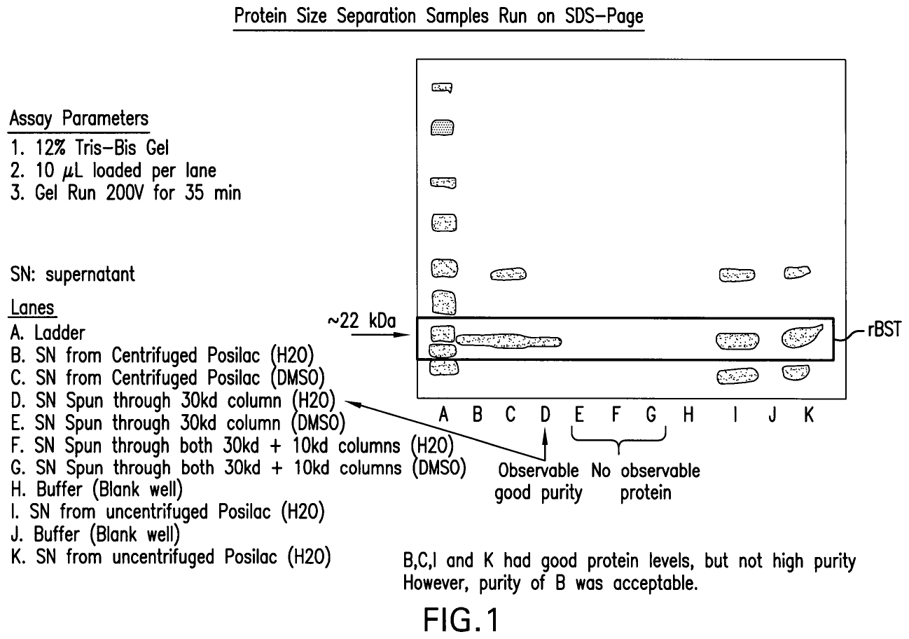

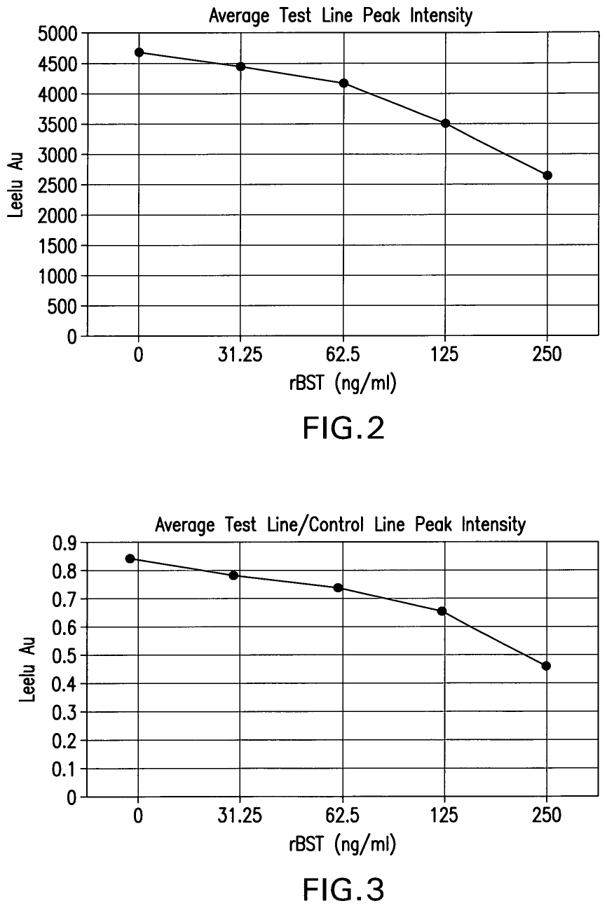

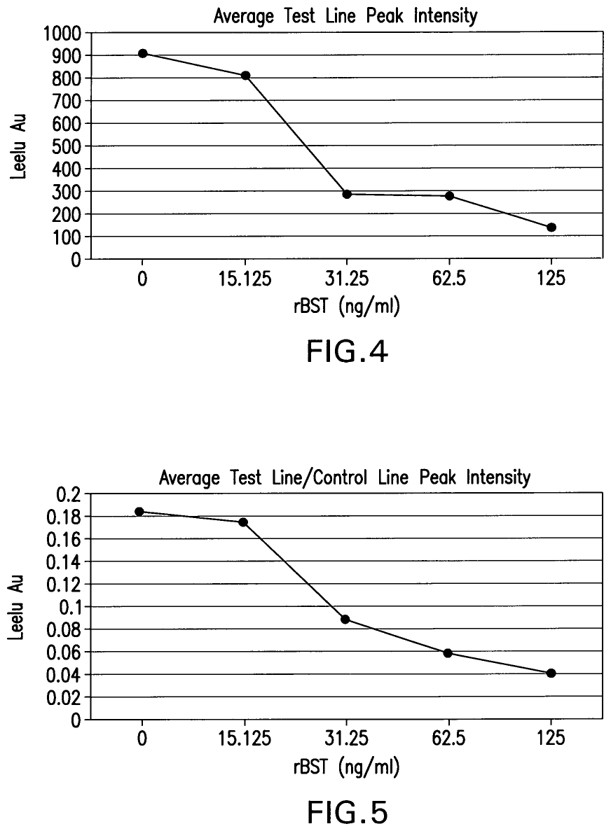

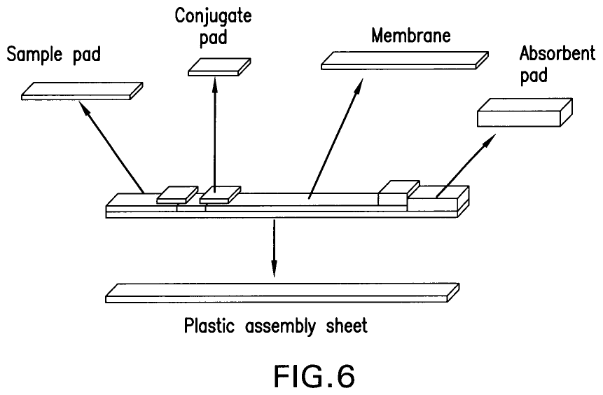

### REFERENCES CITED

Ludwig, S. K. J., Smits, N. G. E., Bremer, M. G. E. G. and Nielen, M. W. F., 2012. Monitoring milk for antibodies against recombinant bovine somatotropin using a microsphere immunoassay-based biomarker approach. Food Control 26: 68-72.
Posthuma-Trumpie, G. A., Korf, J. & van Amerongen, ALateral flow (immuno)assay: its strengths, weaknesses, opportunities and threats. A literature survey. Anal. Bioanalytical Chem. 393:569:582.
Hamlin, D. M. and Walker A. USPTO #U.S. Pat. No. 5,849,499A
Spitsberg, V and Gorewit, R. C. USPTO #U.S. Pat. No. 55,635,401
Safronova, V. A., Samsonova, J. V., Grigorenko, V. G., Osipov A. P. Lateral flow immunoassay for progesterone detection. (2012) Moscow Chemistry University Bulletin 67: 5.Samsonova,
Safronova, V. A Osipov A. P. Pretreatment-free lateral flow enzyme immunoassay for progesterone. Pretreatment-free lateral flow enzyme immunoassay for progesterone detection in whole cows' milk (2015) Talanta 132:685-689.
Mishra, A., Goswami, T. K. and Shukla, D. C. An enzyme-linked immunosorbant assay (ELISA) to measure growth hormone level in serum and milk of buffaloes (Bubalus bubalis). (2007) Indian J. Expt. Biol. 45:594-598.
Samosonova, J. V. Safronova, V. A., Osipov, A. P. Pretreatment-free lateral flow immunoassay enzyme immunoassay for progesterone detection in whole cows's milk (2015) Talanta 132:685-689.
Catigliego, A., Iannoe, G., Grrifoni, G., Rosati, R., Gianfaldoni, D., and Abssandra G. Natural and recombinant bovine somatotropin:immunodetection with a sandwhich ELISA. (2007) J. Dairy Research 74 (1). 79-87.J.V.
Suárez-Pantaleón, C., Huet, A. C., Kavanagh, O., Lei, H., Dervilly-Pinel, G., Le Bizec, B., . . . Delahaut, P. Production of polyclonal antibodies directed to recombinant methionyl bovine somatotropin (2013) Analytica Chemica Acta 76: 186-193.
Chan J. M. Stampfer, M. J., Giovannucci, Gan, P. H., Ma Jing, Wilkinson, P., Hennekens, C. H., Pollack, Plasma Insulin-Like Growth Factor-I and Prostate Cancer Risk: A Prospective Study. (1998a) Science 279:563-566.
Ehard, M. H. et al. (1994) J. of Immunology 15 (1): 1-9 Mishra, A., Goswami, T. K. and Shukla D. C. An Enzyme-Linked immunosorbent assay (ELISA) to measure growth hormone level in serum and milk of buffaloes (Bubalus bubalis). (2007) Indian J. Expt. Biol. 45:594-598.
Hansen, M. (2003) Organic Consumers Organization rBGH and Monsantos Recent intimidation Tactics.
Chan J. M., Giovannucci, E., Andersson,S. O., Yuen j., Adami, H. O. and Wolk, A. Dairy products, calcium, phosphorous, vitamin D, and risk of prostate cancer. Cancer (Sweden). Cancer Causes Control 9:559-566.

What is claimed is:

1. An immunological method of determining whether a lactating cow is being treated with recombinant bovine somatotropin (rbSt).
a. Providing the production of an antigen to produce specific monoclonal mouse/rat antibody(s) against rbSt.
b. Providing a specific monoclonal antibody(s) specific for the analyte rbSt.
c. Providing a process whereby milk can be handled and processed, so that it can be tested for the presence of rbSt in milk.
d. Providing a lateral-flow immunodiffusion platform for detection of rbSt in milk.
e. Providing a process for milk sample analysis. The milk sample is either undiluted or diluted before exposing it to the immunoassay. The cream may be removed from the top of the milk before exposing the sample to the immunoassay. In one embodiment, the milk sample is treated to remove at least 50% of the fat and/or casein. The treatment may be by adding detergent to remove fat globules and/or precipitating the casein with acid. The monoclonal antibody (s) to be used is specific to the n-terminal methionine of the rbSt and therefore does not cross react with the native pituitary (pb St).

2. An immunological method of determining whether a thawed milk sample, dairy product, i.e. cheese, milk powder or dairy derived extract contains recombinant bovine somatotropin (rbSt) comprising the steps of:
a. Providing a sample of frozen then thawed milk or its derivatives from a lactating animal treated with rbSt to be tested.
b. The proposed assay will detect the presence of rbSt in whole milk, pasteurized milk, whole milk and pasteurized milk in solution/diluents, whole milk, pasteurized milk extracts and extracts of processed dairy products.
c. An immune-analytical device for the rapid and simultaneous detection of recombinant bovine somatotropin (rbSt). The method is based on a lateral flow immunoassay technique performed to detect the antigen specific for rbSt in milk or derivatives of milk containing products (dairy products).
d. The method comprising of: Exposing said milk sample to a test membrane having monoclonal antibody that is specific for the antigenic determinant that is the n-terminal amino acid, methionine, of rbSt.

3. The method of claim 1 wherein the cream is removed from the top of the milk before exposing.

4. The method of claim 1 wherein said milk sample is treated to remove at least 50% of the fat and/or the casein.

5. The method of claim 1 wherein said treatment comprises adding a detergent to remove milk fat globules and precipitating the casein with acid.

6. A method whereby exposed milk sample or milk component from said dairy product is exposed to a lateral flow immune diffusion platform described herein for the detection of rb St.
